(19) United States
(12) Patent Application Publication
(10) Pub. No.: US 2024/0335278 A1
(43) Pub. Date: Oct. 10, 2024

(54) EXPANDED FOCAL DEPTH TYPE IMPLANTABLE CONTACT LENS AND PREPARATION METHOD THEREOF

(71) Applicant: WUXI VISION PRO LTD., Wuxi (CN)

(72) Inventors: Shasha LI, Wuxi (CN); Xiugao LIAO, Wuxi (CN); Zhenyu FENG, Wuxi (CN)

(73) Assignee: WUXI VISION PRO LTD., Wuxi (CN)

(21) Appl. No.: 18/746,053

(22) Filed: Jun. 18, 2024

Related U.S. Application Data
(63) Continuation of application No. PCT/CN2023/132566, filed on Nov. 20, 2023.

(30) Foreign Application Priority Data
Dec. 8, 2022 (CN) 202211575612.4

(51) Int. Cl. A61F 2/16

(52) U.S. Cl. CPC A61F 2/1624; A61F 2/1648; A61F 2002/1681; A61F 2240/002; A61F 2240/008

(57) ABSTRACT

An expanded focal depth type implantable contact lens and a preparation method thereof are provided. The expanded focal depth type implantable contact lens includes an optical body, a first supporting loop and a second supporting loop, where the optical body, the first supporting loop and the second supporting loop are of an integrated structure, are made of the same material and are integrally formed, and the optical body is located between the first supporting loop and the second supporting loop; and the optical body is composed of two optical surfaces, one optical surface is a plane, and the other optical surface is a free-form surface with a focal depth expanding function. In the expanded focal depth type implantable contact lens, a focal depth expansion technology is applied to a lens intraocular lens; a focal depth is expanded; and certain adjusting capacity is provided while myopia is corrected.

### CROSS REFERENCE TO THE RELATED APPLICATIONS

This application is the continuation application of International Application No. PCT/2023/132566, filed on Nov. 20, 2023, which is based upon and claims priority to Chinese Patent Application No. 202211575612.4, filed on Dec. 8, 2022, the entire contents of which are incorporated herein by reference.

### TECHNICAL FIELD

The present invention relates to the technical field of optical elements, in particular to an expanded focal depth type implantable contact lens and a preparation method thereof.

### BACKGROUND

An implantable contact lens is an implantable collamer lens (ICL), i.e., an intraocular lens, placed in the posterior chamber.

Due to poor eye care habits of people, long-term use of electronic devices, less outdoor activities and other factors, as well as long-term close eye use cause abnormal growth of the eye axis, thus causing refractive errors. With the development of science and technology and the increasing demand for vision in human beings, many correction methods have been developed to restore the ability of patients to regain their visions. Compared with a corneal laser surgery, traditional framed glasses, contact lenses and other correction methods, intraocular lenses are chosen by more and more young patients because they do not damage the cornea, do not need to be removed, and can be removed and replaced repeatedly.

However, with the increase of age, the ability of ciliary muscle adjustment of myopia patients has weakened, so presbyopia appears, resulting in patients suffering from both myopia and presbyopia. Existing products or methods (e.g., framed glasses, contact lenses, laser corneal surgery, and ICL implantation) for correcting refractive errors such as myopia, presbyopia and astigmatism cannot correct myopia and presbyopia at the same time.

Accordingly, how to design a lens intraocular lens that solves the problems of myopia and presbyopia at the same time and improve the visual quality of patients is a technical problem that needs to be solved by those skilled in the art.

### SUMMARY

The present invention provides an expanded focal depth type implantable contact lens and a preparation method thereof, which have the ability to adjust focal depth expansion, and to solve the problems of both myopia and presbyopia at the same time.

In order to achieve the above effects, the technical solution of the present invention is summarized as follows.

An expanded focal depth type implantable contact lens includes an optical body, a first supporting loop and a second supporting loop, wherein the optical body, the first supporting loop and the second supporting loop are of an integrated structure, are made of the same material and are integrally formed, and the optical body is located between the first supporting loop and the second supporting loop; and said optical body is composed of two optical surfaces that one optical surface is a plane, and the other optical surface is a free-form surface with a focal depth expanding function.

Further, one of said optical surfaces of the optical body satisfies a free-form surface design principle in which a determination method is as follows: an arbitrary spatial rectangular coordinate system is established by taking a vertex of said optical surface as an origin O and an optical axis as a Z-axis, and a coordinate axis X and a coordinate axis Y of the coordinate system are tangent to said free-form surface.

Further, a design process of said free-form surface is as follows:
the free-form surface is divided into a group of sub-surfaces according to a geometric iteration method, such that the free-form surface is discretized, the discretized free-form surface being represented by an envelope surface of the group of sub-surfaces; said free-form surface is rotationally symmetrical, and a sub-surface on one meridian is discussed separately to replace all the sub-surfaces;
it is known that a sub-surface A₀ on the free-form surface has an incident ray vector \vec{I}₀(sin θ₀, cos θ₀) and a normal vector \vec{N}ₐ(−sin γₐ, cos γ₀) respectively, wherein coordinates of a center point of the sub-surface A₀ are (y₀, z₀), θ₀ is an included angle between an incident ray and an optical axis, and γ₀ is an included angle between a normal direction of the sub-surface A₀ and the optical axis; when the incident ray is parallel light, that is, θ₀-0°, the incident ray vector is \vec{I}₀(0, 1).
the incident ray is focused to a focal point F on the optical axis after being refracted through the sub-surface A₀, coordinates of the focal point F are (0, f₀), f₀ is a focal length of the focal point F, and an included angle θ₁ between an emergent ray of the sub-surface A₀ and the optical axis is obtained according to a geometric relationship between a focal position of the sub-surface A₀ and the optical axis, θ₁ being characterized by an equation:

$${θ}_{1} = arcsin ( \frac{{𝓎}_{0}}{{f}_{0} - {z}_{0}} ) \qquad ( 1 )$$

the incident ray and the optical axis satisfy the following relationship:

$$\{ \begin{matrix}{θ}_{1} + {γ}_{0} = {β}_{2} \\ {β}_{1} = {γ}_{0}\end{matrix} \qquad ( 2 )$$

in which: β₁ is an incident angle of the incident ray passing through the intraocular lens, β₂ is an emergent angle of the emergent ray \vec{I}₁ passing through the intraocular lens, that is, the incident angle is an included angle between the incident ray and a normal of the sub-surface A₀, and the emergent angle is an included angle between the emergent ray \vec{I}₁ and the normal of the sub-surface A₀;
according to the Snell's law:

$${n}_{1} sin {β}_{1} = {n}_{2} sin {β}_{2} \qquad ( 3 )$$

in which: n₁ and n₂ are refractive indices of an incident medium and an emergent medium respectively, the refractive indices of the incident medium and the emergent medium are known quantities, and the incident medium represents the intraocular lens; an included angle γ₀ between the normal direction of the sub-surface A₀ and the optical axis is solved according to simultaneous equations (2) and (3), γ₀ being expressed as:

$${γ}_{0} = arctan ( \frac{sin {θ}_{1}}{\frac{{n}_{1}}{{n}_{2}} - cos {θ}_{1}} ) \qquad ( 4 )$$

a normal vector \vec{N}₀(−sin γ₀, cos γ₀) of the sub-surface A₀ is obtained according to γ₀; the normal vectors of all sub-surfaces are iteratively solved in sequence; and then, an envelope surface, i.e., a free-form surface, of all the sub-surfaces is fitted.

In the above scheme, the intraocular lens of the present invention has a plurality of focal points, and a long focal depth range.

An emergent ray \vec{I}₁ obtained after an incident ray is refracted by passing through the sub-surface A₀ may be obtained according to θ₁, wherein \vec{I}₁ is characterized by an equation as \vec{I}₁=C|\vec{I}₀|(sin θ₁,cos θ₁), in which C is a transmittance of the intraocular lens.

Further, an effective optical zone of said optical body has a diameter ranging from 4 mm to 6 mm.

Further, the focal length f₀ of said optical body is determined by a dioptric power of the optical body, and the dioptric power ranges from 0 D to −30 D.

Further, said optical body is made of hydrophilic polyacrylate.

Further, said optical body has a refractive index of 1.437 at 35° C.

Further, said first supporting loop and said second supporting loop each have a thickness of 0.08 mm to 0.15 mm.

A preparation method of an expanded focal depth type implantable contact lens is used to design the expanded focal depth type implantable contact lens, the preparation method including the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface that satisfies a dioptric power requirement of the optical body, and calculating a focal length range fₘᵢₙ to fₘₐₓ of the optical body according to the dioptric power of the optical body,

$$\begin{matrix}{f}_{min} = 1 / ϕ \\ {f}_{max} = 1 / ( ϕ + {ϕ}_{expanded} )\end{matrix} \qquad ( 5 )$$

in which: fₘᵢₙ and fₘₐₓ are a minimum focal length and a maximum focal length of the optical body (1) respectively, and ϕ and ϕ_{expanded} are the dioptric power and an expanded dioptric power of the optical body (1) respectively;
setting a number i of geometric iterations to discretize the optimized basic spherical surface, wherein the number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body (1) in sequence by using the geometric iteration method, and fitting an envelope surface, i.e., a free-form surface, of all sub-surfaces A₀ in combination with point coordinates of the sub-surface A₀;
S2: turning-milling machining: compiling a lathe program of a hydrophilic material according to the free-form surface designed by step S1; turning the optical body by using a diamond single-point cutting technology; compiling a milling machine program, and milling an appearance of an optic of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

It may be understood that the normal vector of the sub-surface passes through the sub-surface and is perpendicular to a tangent plane of the sub-surface, the sub-surface is a miniature spherical surface, and the sub-surface may be solved by means of a geometric relationship of the normal vectors of the sub-surface. In step 1, a curvature r of the basic spherical surface that satisfies a dioptric power requirement of the optical body is optimized, wherein the optimization is completed by adjusting a parameter of the basic spherical surface by zemax.

The sub-surface A₁ has a corresponding focal length of f₁, and the sub-surface Aᵢ has a corresponding focal length of fᵢ.

Compared with the prior art, the technical solutions of the present invention have the following beneficial effects.

According to the present invention, a focal length expansion technology is applied to phakic intraocular lens, so that a focal depth adjusting capacity of the lens is increased on the basis of correcting myopia.

1. According to the present invention, the free-form surface is introduced; the focal depth expansion technology is applied to phakic intraocular lens; a focal depth is expanded; certain adjusting capacity is provided while myopia is corrected; the surface is smooth and does not change suddenly; and glare and halo cannot be introduced, such that presbyopia of a patient is corrected while myopia is corrected, thereby achieving an excellent visual effect.

2. According to the present invention, a spherical or aspheric surface of the ordinary phakic intraocular lens is replaced with the free-form surface, which effectively controls the lens thickness, and is more universal.

3. The phakic intraocular lens of the present invention is formed integrally, has a simple structure and good stability, is suitable for complex ophthalmic fluid environments, and is not easy to induce complications.

### BRIEF DESCRIPTION OF THE DRAWINGS

The accompanying drawings are for an illustrative purpose only and cannot be construed as limitations on the present invention. In order to better illustrate the embodiments, certain components in the accompanying drawings will be omitted, increased or reduced, and do not represent the dimension of an actual product. For those skilled in the art, it may be understood that certain well-known structures and their descriptions in the accompanying drawings may be omitted.

FIG. 1 is a schematic structural diagram of a front surface of a phakic intraocular lens provided by an embodiment of the present invention;

FIG. 2 is a schematic structural diagram of a side surface of the phakic intraocular lens provided by an embodiment of the present invention;

FIG. 3 is an optical path diagram of a sub-surface provided by an embodiment of the present invention; and

FIG. 4 is a schematic diagram of focal depth expansion provided by an embodiment of the present invention.

### DETAILED DESCRIPTION OF THE EMBODIMENTS

In order to make the objectives, technical solutions and advantages of the embodiments of the present invention more clearly, the technical solutions in the embodiments of the present invention will be described clearly and completely in conjunction with the accompanying drawings in the embodiments of the present invention. Apparently, the described embodiments are merely some embodiments, rather than all embodiments, of the present invention. Based on the embodiments of the present invention, all other embodiments derived by a person of ordinary skill in the art without creative efforts shall fall within the protection scope of the present invention.

### Embodiment 1

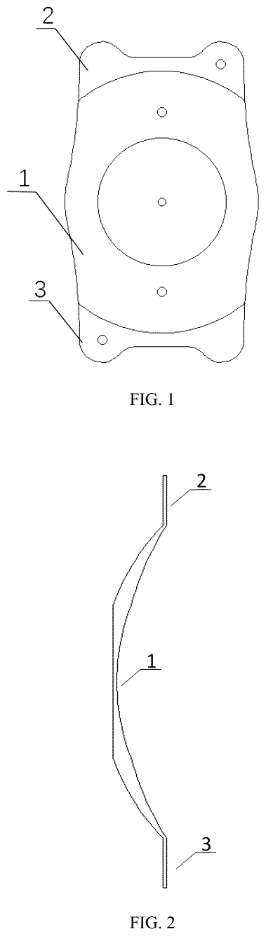

For ease of understanding, referring to FIG. 1, the present invention provides an embodiment of an expanded focal depth type implantable contact lens. The expanded focal depth type implantable contact lens includes an optical body 1, a first supporting loop 2 and a second supporting loop 3, wherein the optical body 1, the first supporting loop 2 and the second supporting loop 3 are of an integrated structure, are made of the same material and are integrally formed, and said optical body 1 is located between the first supporting loop 2 and the second supporting loop 3; and said optical body 1 is composed of two optical surfaces, one optical surface is a plane, and the other optical surface is a free-form surface with a focal depth expanding function.

The free-form surface is divided into a group of sub-surfaces according to a geometric iteration method, such that the free-form surface is discretized, the discretized free-form surface being represented by an envelope surface of the group of sub-surfaces; said free-form surface is rotationally symmetrical, and a sub-surface on one meridian is discussed separately to replace all the sub-surfaces;
it is known that a sub-surface A₀ on the free-form surface has an incident ray vector \vec{I}₀(sin θ₀, cos θ₀) and a normal vector \vec{N}₀(−sin γ₀, cos γ₀) respectively, wherein coordinates of a center point of the sub-surface A₀ are (y₀, z₀), θ₀ is an included angle between an incident ray and an optical axis, and γ₀ is an included angle between a normal direction of the sub-surface A₀ and the optical axis; when the incident ray is parallel light, that is, θ₀=0°, the incident ray vector is \vec{I}₀(0, 1).
the incident ray is focused to a focal point F on the optical axis after being refracted through the sub-surface A₀, coordinates of the focal point F are (0, f₀), f₀ is a focal length of the focal point F, and an included angle θ₁ between an emergent ray of the sub-surface A₀ and the optical axis is obtained according to a geometric relationship between a focal position of the sub-surface A₀ and the optical axis, θ₁ being characterized by an equation:

$${θ}_{1} = arcsin ( \frac{{𝓎}_{0}}{{f}_{0} - {z}_{0}} ) \qquad ( 1 )$$

the incident ray and the optical axis satisfy the following relationship:

$$\{ \begin{matrix}{θ}_{1} + {γ}_{0} = {β}_{2} \\ {β}_{1} = {γ}_{0}\end{matrix} \qquad ( 2 )$$

in which: β₁ is an incident angle of the incident ray passing through the intraocular lens, β₂ is an emergent angle of the emergent ray \vec{I}ᵢ passing through the intraocular lens, that is, the incident angle is an included angle between the incident ray and a normal of the sub-surface A₀, and the emergent angle is an included angle between the emergent ray \vec{I}₁ and the normal of the sub-surface A₀;
according to the Snell's law:

$${n}_{1} sin {β}_{1} = {n}_{2} sin {β}_{2} \qquad ( 3 )$$

in which: n₁ and n₂ are refractive indices of an incident medium and an emergent medium respectively, the refractive indices of the incident medium and the emergent medium are known quantities, and the incident medium represents the intraocular lens; an included angle γ₀ between the normal direction of the sub-surface A₀ and the optical axis is solved according to simultaneous equations (2) and (3), γ₀ being expressed as:

$${γ}_{0} = arctan ( \frac{sin {θ}_{1}}{\frac{{n}_{1}}{{n}_{2}} - cos {θ}_{1}} ) \qquad ( 4 )$$

a normal vector \vec{N}₀(−sin γ₀, cos γ₀) of the sub-surface A₀ is obtained according to γ₀; the normal vectors of all sub-surfaces are iteratively solved in sequence; and then, an envelope surface, i.e., a free-form surface, of all the sub-surfaces is fitted.

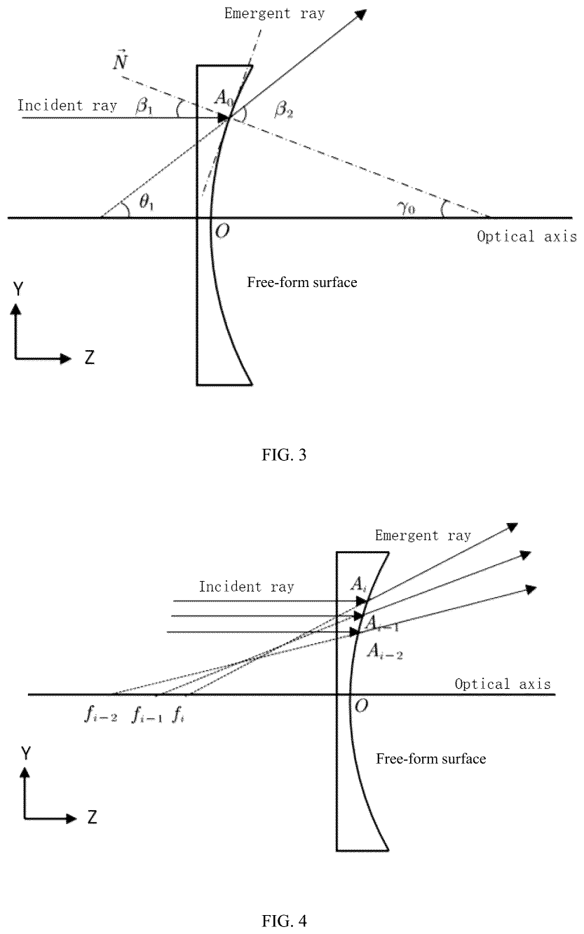

It should be noted that the included angle γ₀ between the normal direction of the sub-surface Aᵢ and the optical axis solved according to simultaneous equations (2) and (3) is solved according to equivalence relationships in the equations (2) and (3). The derivation process is as shown in FIG. 3:

$$\begin{matrix}∵ {β}_{1} = {γ}_{0} \\ ∴ {n}_{1} sin {β}_{1} = {n}_{1} sin {γ}_{0} = {n}_{2} sin {β}_{2} \\ ∵ {θ}_{1} + {γ}_{0} = {β}_{2} \\ ∴ {n}_{1} sin {γ}_{0} = {n}_{2} sin ( {θ}_{1} + {γ}_{0} ) \\ ∴ \frac{{n}_{1}}{{n}_{2}} sin {γ}_{0} = sin {θ}_{1} cos {γ}_{0} + sin {γ}_{0} cos {θ}_{1} \\ ∴ ( \frac{{n}_{1}}{{n}_{2}} - cos {θ}_{1} ) sin {γ}_{0} = sin {θ}_{1} cos {γ}_{0} \\ ∴ \frac{sin {γ}_{0}}{cos {γ}_{0}} = tan {γ}_{0} = \frac{sin {θ}_{1}}{\frac{{n}_{1}}{{n}_{2}} - cos {θ}_{1}} \\ ∴ {γ}_{0} = arctan ( \frac{sin {θ}_{1}}{\frac{{n}_{1}}{{n}_{2}} - cos {θ}_{1}} )\end{matrix}$$

specifically, said optical body 1 is made of hydrophilic polyacrylate, and has a refractive index of 1.437 at 35° C.

A preparation method of an expanded focal depth type implantable contact lens is used to design the expanded focal depth type implantable contact lens, the preparation method including the following steps:
S1: optical design: determining a dioptric power, an effective optical zone diameter and an expanded focal depth value of the optical body 1; modeling in zemax, taking a free-form surface before discretization as a basic spherical surface, optimizing a curvature r of the basic spherical surface that satisfies a dioptric power requirement of the optical body 1, and calculating a focal length range fₘᵢₙ to fₘₐₓ of the optical body 1 according to the dioptric power of the optical body 1,

$$\begin{matrix}{f}_{min} = 1 / ϕ \\ {f}_{max} = 1 / ( ϕ + {ϕ}_{expanded} )\end{matrix} \qquad ( 5 )$$

in which: fₘᵢₙ and fₘₐₓ are a minimum focal length and a maximum focal length of the optical body 1 respectively, and ϕ and ϕ_{expanded} are the dioptric power and an expanded dioptric power of the optical body 1 respectively;
setting a number i of geometric iterations to discretize the optimized basic spherical surface, wherein a number of sub-surface A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fi increasing in sequence, wherein in order not to cause interference to an incident ray, pupil scaling is taken into account, pupil dependence is reduced, and the absolute value of the focal length fᵢ of the sub-surface Aᵢ increases from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body 1 in sequence by using the geometric iteration method, and fitting an envelope surface, i.e., a free-form surface, of all sub-surfaces A₀ in combination with point coordinates of the sub-surface A₀;
S2: turning-milling machining: compiling a lathe program of a hydrophilic material according to the free-form surface designed by step S1; turning the optical body 1 by using a diamond single-point cutting technology; compiling a milling machine program, and milling an appearance of an optical zone of the optical body 1 and legs of the first supporting loop 2 and the second supporting loop 3;
S3: polishing treatment: performing barrel polishing on the intraocular lens at a low temperature; and
S4: test validation: analyzing and testing the intraocular lens in a simulated eye system.

According to the present invention, the free-form surface is introduced; a focal depth expansion technology is applied to the phakic intraocular lens; a focal depth is expanded; certain adjusting capacity is provided while myopia is corrected; the surface is smooth and does not change suddenly; and glare and halo are not introduced, such that presbyopia of a patient is corrected while myopia is corrected, and thus the patient can achieve a clear vision.

It should be noted that presbyopia is caused by the weakening of the patient's ability to adjust the lenses of the human eyes. The lens of the present invention provides a part of the adjusting capacity to correct the presbyopia. The adjusting capacity allows patients to still see clearly in a relatively large range of dioptric power (+1 D to +2.5 D) at an expanded focal length with fully correcting farsighted vision and without using an adjusting function of own lenses of the human eyes.

### Embodiment 2

Specifically, on the basis of Embodiment 1, the scheme is explained in combination with specific embodiments, and the technical effect of this scheme is further reflected. Specifically:
a preparation method of an expanded focal depth type implantable contact lens in the present embodiment includes:
(1) design scheme: determining that a dioptric power of the intraocular lens of −10 D, an optic diameter of 5 mm, and an expanded focal depth of −1 D;
(2) optical design: modeling in zemax through the fitted dioptric power and optic area (determined by the optical zone diameter) of the intraocular lens, optimizing a curvature r=10.4 mm of a basic spherical surface, which is a concave surface, and calculating a focal length ranging from −100 mm to −111.11 mm according to the equation (5); setting a number of geometric iterations to 100, and discretizing the basic spherical surface, wherein the number of sub-surfaces on one meridian is 100 due to the rotational symmetry of the intraocular lens; calculating a center position of a group of sub-surfaces, A₁ (y₁, z₁)˜A₁₀₀ (y₁₀₀, z₁₀₀) from inside to outside respectively; and dividing the focal length range as:

$$\begin{matrix}{f}_{1} = - 100 mm \\ {f}_{2} = - 100.11 mm \\ {f}_{3} = - 100.22 mm \\ … \\ {f}_{100} = - 111.11 mm\end{matrix}$$

in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body 1 in sequence by using the geometric iteration method, and fitting an envelope surface (as shown in FIG. 4), i.e., a free-form surface, of all sub-surfaces A₀ in combination with point coordinates of the sub-surface A₀;
(3) turning-milling machining: compiling a lathe program of a hydrophilic material according to the free-form surface; turning the optical body by using a diamond single-point cutting technology; compiling a milling machine program and milling an appearance of an optic of the optical body 1 and legs of the first supporting loop 2 and the second supporting loop 3;
(4) polishing treatment: performing barrel polishing at a low temperature to obtain an intraocular lens with a qualified optical surface; and
(5) test validation: analyzing and testing the intraocular lens in a simulated eye system.

In the present embodiment, the phakic intraocular lens is introduced into an ocular model required in ISO11979-2, and an out-of-focus MTF value is obtained by means of testing with an optical device.

### Embodiment 3

Specifically, on the basis of Embodiment 1, the scheme is explained in combination with specific embodiments, and the technical effect of this scheme is further reflected. Specifically:

A preparation method of an expanded focal depth type implantable contact lens in the present embodiment includes:
(1) design scheme: determining that a dioptric power of the intraocular lens of −15 D, an optic diameter of 5.5 mm, and an expanded focal depth of −1.5 D;
(2) optical design: modeling in zemax through the fitted dioptric power and optic diameter of the intraocular lens, optimizing a curvature r=6.93 mm of a basic spherical surface, which is a concave surface, and calculating a focal length ranging from −66.67 mm to −60.6 mm according to the equation (5); setting a number of geometric iterations to 150, and discretizing the basic spherical surface, wherein the number of sub-surfaces on one meridian is 150 due to the rotational symmetry of the lens; calculating a center position of a group of sub-surfaces, A₁ (y₁, z₁)˜A₁₅₀ (y₁₅₀, z₁₅₀) from inside to outside respectively; and dividing the focal length range as:

$$\begin{matrix}{f}_{1} = - 60.6 mm \\ {f}_{2} = - 60.646 mm \\ {f}_{3} = - 60.687 mm \\ … \\ {f}_{150} = - 66.667 mm\end{matrix}$$

in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body 1 in sequence by using the geometric iteration method, and fitting an envelope surface (as shown in FIG. 4), i.e., a free-form surface, of all sub-surfaces A₀ in combination with point coordinates of the sub-surface A₀;
(3) turning-milling machining: compiling a lathe program of a hydrophilic material according to the free-form surface; turning the optical body by using a diamond single-point cutting technology; compiling a milling machine program and milling an appearance of an optic of the optical body 1 and legs of the first supporting loop 2 and the second supporting loop 3;
(4) polishing treatment: performing barrel polishing at a low temperature to obtain an intraocular lens with a qualified optical surface; and
(5) test validation: analyzing and testing the intraocular lens in a simulated eye system.

In the present embodiment, the phakic intraocular lens is introduced into an ocular model required in ISO11979-2, and an out-of-focus MTF value is obtained by means of testing with an optical device.

### Embodiment 4

Specifically, on the basis of Embodiment 1, the scheme is explained in combination with specific embodiments, and the technical effect of this scheme is further reflected. Specifically:

A preparation method of an expanded focal depth type implantable contact lens in the present embodiment includes:
(1) design scheme: determining that a dioptric power of the intraocular lens of −20 D, an optic diameter of 5 mm, and an expanded focal depth of −1.75 D;
(2) optical design: modeling in zemax through the fitted dioptric power and optic diameter of the intraocular lens, optimizing a curvature r=5.2 mm of a basic spherical surface, which is a concave surface, and calculating a focal length ranging from −50 mm to −45.98 mm according to the equation (5); setting a number of geometric iterations to 175, and discretizing the basic spherical surface, wherein the number of sub-surfaces on one meridian is 175 due to the rotational symmetry of the lens; calculating a center position of a group of sub-surfaces, A₁ (y₁, z₁)−A₁₇₅ (y₁₇₅, z₁₇₅) from inside to outside respectively; and dividing the focal length range as:

$$\begin{matrix}{f}_{1} = - 45.977 mm \\ {f}_{2} = - 46 mm \\ {f}_{3} = - 46.023 mm \\ … \\ {f}_{175} = - 50 mm\end{matrix}$$

in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body 1 in sequence by using the geometric iteration method, and fitting an envelope surface (as shown in FIG. 4), i.e., a free-form surface, of all sub-surfaces A₀ in combination with point coordinates of the sub-surface A₀;
(3) turning-milling machining: compiling a lathe program of a hydrophilic material according to the free-form surface; turning the optical body by using a diamond single-point cutting technology; compiling a milling machine program and milling an appearance of an optic of the optical body 1 and legs of the first supporting loop 2 and the second supporting loop 3;
(4) polishing treatment: performing barrel polishing at a low temperature to obtain an intraocular lens with a qualified optical surface; and
(5) test validation: analyzing and testing the intraocular lens in a simulated eye system.

In the present embodiment, the phakic intraocular lens is introduced into an ocular model required in ISO11979-2, and an out-of-focus MTF value is obtained by means of testing with an optical device.

It may be understood that the center point and the normal vector in the above embodiment are represented by two points, which refer to a coordinate representation in a coordinate system Y-Z, and at this time, X coordinates are omitted.

Obviously, the above embodiments of the present invention are only examples given to clearly illustrate the present invention, without any limitation of implementations of the present invention. For a person of ordinary skill in the art, other different forms of changes or variations can be made on the basis of the above description. There is no need and cannot be exhaustive of all implementations. Any modification, equivalent replacement, improvement and so on made within the spirit and principle of the present invention shall be encompassed by the protection scope of the present invention.

1. An expanded focal depth type implantable contact lens, comprising an optical body, a first supporting loop and a second supporting loop, wherein the optical body, the first supporting loop and the second supporting loop are of an integrated structure, are made of a same material and are integrally formed, and the optical body is located between the first supporting loop and the second supporting loop; the optical body comprises a first optical surface and a second optical surface, wherein the first optical surface is a plane, and the second optical surface is a free-form surface with a focal depth expanding function.

2. The expanded focal depth type implantable contact lens according to claim 1, wherein the second optical surface of the optical body satisfies a free-form surface design principle, wherein a determination method in the free-form surface design principle is as follows: a spatial rectangular coordinate system is established by taking a vertex of the second optical surface as an origin O and an optical axis as a Z-axis, and a coordinate axis X and a coordinate axis Y of the spatial rectangular coordinate system are tangent to the free-form surface.

3. The expanded focal depth type implantable contact lens according to claim 2, wherein a design process of the free-form surface is as follows:
the free-form surface is divided into a group of sub-surfaces according to a geometric iteration method, such that the free-form surface is discretized, the free-form surface discretized being represented by an envelope surface of the group of sub-surfaces; the free-form surface is rotationally symmetrical, and a sub-surface on one meridian is discussed separately to represent all the sub-surfaces;
a sub-surface A₀ on the free-form surface has an incident ray vector I₀(sin θ₀, cos θ₀) and a normal vector \vec{N}₀(−sin γ₀, cos γ₀) respectively, wherein coordinates of a center point of the sub-surface A₀ are (y₀, z₀), θ₀ is an included angle between an incident ray and an optical axis, and γ₀ is an included angle between a normal direction of the sub-surface A₀ and the optical axis; when the incident ray is parallel light, that is, θ₀-0°, the incident ray vector is \vec{I}₀(0, 1);
the incident ray is focused to a focal point F on the optical axis after being refracted through the sub-surface A₀, coordinates of the focal point F are (0, f₀), f₀ is a focal length of the focal point F, and an included angle θ₁ between an emergent ray of the sub-surface A₀ and the optical axis is obtained according to a geometric relationship between a focal position of the sub-surface A₀ and the optical axis, θ₁ being characterized by an equation:
$${θ}_{{1}^{=}} arc sin ( \frac{{y}_{0}}{{f}_{0} - {z}_{0}} ) \qquad ( 1 )$$
the incident ray and the optical axis satisfy the following relationship:
$$\{ \begin{matrix}{θ}_{1} + {γ}_{0} = {β}_{2} \\ {β}_{1} = {γ}_{0}\end{matrix} \qquad ( 2 )$$
wherein β₁ is an incident angle of the incident ray passing through an intraocular lens, and β₂ is an emergent angle of the emergent ray \vec{I}₁ passing through the intraocular lens, wherein the incident angle is an included angle between the incident ray and a normal of the sub-surface A₀, and the emergent angle is an included angle between the emergent ray \vec{I}₁ and the normal of the sub-surface A₀;
according to the Snell's law:
n₁ sinβ₁=n₂ sinβ₂ (3)
wherein n₁ and n₂ are a refractive index of an incident medium and a refractive index of an emergent medium respectively, the refractive index of the incident medium and the refractive index of the emergent medium are known quantities, and the incident medium represents the intraocular lens; an included angle γ₀ between the normal direction of the sub-surface A₀ and the optical axis is solved according to simultaneous equations (2) and (3), γ₀ being expressed as:
$${γ}_{0} = arctan ( \frac{sin {θ}_{1}}{\frac{{n}_{1}}{{n}_{2}} - cos {θ}_{1}} ) \qquad ( 4 )$$
a normal vector \vec{N}₀(−sin γ₀, cos γ₀) of the sub-surface A₀ is obtained according to γ₀; normal vectors of all sub-surfaces are iteratively solved in sequence; and then, an envelope surface of all the sub-surfaces is fitted to obtain the free-form surface.

4. The expanded focal depth type implantable contact lens according to claim 1, wherein the focal length f₀ of the optical body is determined by a dioptric power of the optical body, and the dioptric power ranges from 0 D to −30 D.

5. The expanded focal depth type implantable contact lens according to claim 1, wherein an effective optical zone of the optical body has a diameter ranging from 4 mm to 6 mm.

6. The expanded focal depth type implantable contact lens according to claim 1, wherein an expanded focal depth of the optical body ranges from −1 D to −2.5 D.

7. The expanded focal depth type implantable contact lens according to claim 1, wherein the optical body is made of hydrophilic polyacrylate.

8. The expanded focal depth type implantable contact lens according to claim 1, wherein the optical body has a refractive index of 1.437 at 35° C.

9. The expanded focal depth type implantable contact lens according to claim 1, wherein each of the first supporting loop and the second supporting loop has a thickness of 0.08 mm to 0.15 mm.

10. A preparation method of the expanded focal depth type implantable contact lens according to claim 1, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface Aᵢ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

11. A preparation method of the expanded focal depth type implantable contact lens according to claim 2, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

12. A preparation method of the expanded focal depth type implantable contact lens according to claim 3, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to the incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

13. A preparation method of the expanded focal depth type implantable contact lens according to claim 4, comprising the following steps:
S1: optical design: determining the dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

14. A preparation method of the expanded focal depth type implantable contact lens according to claim 5, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ (−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

15. A preparation method of the expanded focal depth type implantable contact lens according to claim 6, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

16. A preparation method of the expanded focal depth type implantable contact lens according to claim 7, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

17. A preparation method of the expanded focal depth type implantable contact lens according to claim 8, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.

18. A preparation method of the expanded focal depth type implantable contact lens according to claim 9, comprising the following steps:
S1: optical design: determining a dioptric power, an effective optical zone diameter and an expanded focal depth value of the optical body; modeling in zemax, taking a free-form surface before discretization as a basic spherical surface, optimizing a curvature r of the basic spherical surface to obtain an optimized basic spherical surface of satisfying a dioptric power requirement of the optical body, and calculating a focal length range fₘᵢₙ to fₘₐₓ of the optical body according to the dioptric power of the optical body,
$${f}_{min} = 1 / ϕ \qquad ( 5 )$$
$${f}_{max} = 1 / ( ϕ + {ϕ}_{e x p a n d e d} )$$
wherein fₘᵢₙ and fₘₐₓ are a minimum focal length and a maximum focal length of the optical body respectively, and ϕ and ϕ_{expanded} are the dioptric power and an expanded dioptric power of the optical body respectively;
setting a number i of geometric iterations to discretize the optimized basic spherical surface, wherein a number of sub-surfaces A₀ on one meridian is i and the intraocular lens is rotationally symmetrical; calculating positions of center points of all sub-surfaces A₀, Aᵢ representing any sub-surface on one meridian, (yᵢ, zᵢ) representing a position of the center point of any sub-surface A₀, and fᵢ representing a focal length of any sub-surface Aᵢ; dividing the focal length range of f₁ to fᵢ, absolute values from f₁ to fᵢ increasing in sequence, wherein in order not to cause interference to an incident ray, pupil scaling is taken into account, pupil dependence is reduced, and the absolute values of the focal lengths fᵢ of the sub-surfaces Aᵢ increase from the center to the outside;
modeling in matlab according to a geometric iteration method, and solving an included angle θᵢ between an emergent ray of the sub-surface Aᵢ and the optical axis according to the focal length fᵢ of the sub-surface Aᵢ and the position Aᵢ(yᵢ, zᵢ) of the center point of the sub-surface;
solving γ₀ according to the equation (4), and obtaining a normal vector \vec{N}ᵢ(−sin γᵢ, cos γᵢ) of the sub-surface Aᵢ through γᵢ, γᵢ being used to represent an included angle between a normal direction of any sub-surface Aᵢ and the optical axis; solving normal vectors of all sub-surfaces A₀ on one meridian of the optical body in sequence by using the geometric iteration method, and fitting an envelope surface of all sub-surfaces A₀ in combination with point coordinates of the sub-surface A₀ to obtain the free-form surface;
S2: turning-milling machining: compiling a lathe program of a hydrophilic material according to the free-form surface designed by step S1; turning the optical body by using a diamond single-point cutting technology; compiling a milling machine program, and milling an appearance of an optical zone of the optical body and legs of the first supporting loop and the second supporting loop;
S3: polishing treatment: performing barrel polishing on the intraocular lens at a low temperature; and
S4: test validation: analyzing and testing the intraocular lens in a simulated eye system.
